(19) United States
(12) Patent Application Publication
(10) Pub. No.: US 2012/0141993 A1
(43) Pub. Date: Jun. 7, 2012

(54) COMPOSITIONS AND METHODS FOR NEISSERIA GONORRHOEAE DIAGNOSTIC TESTING

(75) Inventors: Mitra Choudhury Singhal, Edmonds, WA (US); Cori Anne Barfield, Seattle, WA (US); Kathryn Watts Weaver, Washington, DC (US)

(73) Assignee: PROGRAM FOR APPROPRIATE TECHNOLOGY IN HEALTH, Seattle, WA (US)

(21) Appl. No.: 13/309,391

(22) Filed: Dec. 1, 2011

Related U.S. Application Data
(60) Provisional application No. 61/418,684, filed on Dec. 1, 2010.

(51) Int. Cl. C12Q 1/68; C07H 21/04

(52) U.S. Cl. 435/6.11; 435/6.12; 536/24.33

(57) ABSTRACT

The invention provides methods, reagent, and kits for detecting the presence of Neisseria gonorrhoeae in a test sample.

### CROSS-REFERENCE TO RELATED APPLICATION

This application claims the benefit of U.S. Provisional Application No. 61/418,684, filed Dec. 1, 2010.

### STATEMENT OF GOVERNMENT LICENSE RIGHTS

This invention was made with Government support under Grant Number U01 AI070801-05 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 38262_Seq_FINAL_{—}2011-12-01.txt. The text file is 8 KB; was created on Dec. 1, 2011; and is being submitted via EFS-Web with the filing of the specification.

### BACKGROUND

Gonorrhea is a bacterial infection of the lower genital tract that is transmitted mainly by sexual contact. Gonorrhea is caused by the bacteria Neisseria gonorrhoeae. Infection causes urethritis in men and cervicitis in women. Ascending infection in women can also lead to the development of acute pelvic inflammatory disease, one of the leading causes of female infertility. Gonorrhea infection can also be passed from an infected mother to her baby during vaginal delivery, and can result in gonococcal conjunctivitis in the newborn's eyes. One important aspect of gonorrheal infections is that they are often asymptomatic. This contributes to further transmission and maintenance of the bacterium within populations, and highlights the need for enhanced and accessible diagnosis and surveillance (“Centers for Disease Control and Prevention: Sexually Transmitted Disease Surveillance, 2003,” U.S. Department of Health and Human Services, Center for Disease Control, Atlanta, Ga., 2003), Greater than 350,000 cases of gonorrhea were reported in the United States by the Center for Disease Control and the World Health Organization estimates 19 million cases occur in the African continent annually, Diagnosis and treatment of gonorrhea are important to prevent the spread of the bacteria, and has become increasingly problematic due to increasing resistance to antibiotics and the observation that infection does not elicit protective immunity. At present, there is no effective vaccine and, as a result, control of gonococcal infections depends on surveillance of at-risk populations and early intervention to treat infected individuals (R. Viscidi, et al, J. Microbiol. 41:197-204, 2003).

Bacterial culture is both sensitive and specific for the detection of gonorrhea and is regarded as a gold standard for definitive laboratory diagnosis of gonorrhea; but the need to collect invasive specimens and to transport specimens in conditions that maintain the viability of organisms is a major limitation of this method. A number of commercially available nucleic acid tests (NAAT) have been developed as sensitive and more rapid methods to diagnose infection. These include the Roche Amplicor® test, targeting the cytosine methyl transferase gene, the Becton Dickinson ProbeTec™ SDA assay targeting the multi-copy pilin (pil C, pil E) proteins, the Abbott LCx® assay, targeting opacity genes (opa), the GenProbe Aptima, Combo 2® test targeting 16S ribosomal RNA gene (16S rRNA), and the Cepheid GeneExpert® assay, Several of these tests for gonorrhea are multiplexed with tests to detect Chlamydia trachomatis.

The design of NAATs for the detection of Neisseria gonorrhoeae faces two major challenges sequence variation between Neisseria gonorrhoeae sub-types, and cross-reaction with other Neisseria species, from which it is speculated Neisseria gonorrhoeae evolved due to recombination events. The Neisseria gonorrhoeae species is made up of organisms with a broad range of sub-types that show considerable genetic variation that are not randomly distributed. The implications of this are that NAATs can perform differently depending on the patient population, geographic distribution, and in the year samples are tested, resulting in false negative results. In addition, the genomes of Neisseria gonorrhoeae and the related Neisseria species, Neisseria meningitis, are highly homologous, making targeting of NAATs challenging (D. Whiley, et al, J. of Molec. Diagnostics 8:3-15, 2006).

Therefore, there remains a need for a test that is both specific and inclusive of different Neisseria gonorrhoeae sub-types and that reduces the number of false positive results due to cross-reactivity with other Neisseria species or other bacteria.

### SUMMARY

In accordance with the foregoing, in one aspect, the invention provides a method for determining the presence of Neisseria gonorrhoeae (“N. gonorrhoeae”) in a test sample. The method according to this aspect of the invention comprises (a) contacting a test sample with a composition comprising at least one primer pair comprising a forward primer and a reverse primer capable of hybridizing to a target region of the N. gonorrhoeae opacity (opa) gene consisting of SEQ ID NO:2 to form a reaction mixture; and (b) subjecting said reaction mixture to amplification conditions suitable to amplify at least a portion of said target region.

In another aspect, the invention provides a method for determining the presence of N. gonorrhoeae in a test sample. The method according to this aspect of the invention comprises (a) contacting a test sample with a composition comprising at least one primer pair comprising a forward primer and a reverse primer capable of hybridizing to a target region of the N. gonorrhoeae porin A (por A) pseudo-gene consisting of SEQ ID NO:7 to form a reaction mixture; and (b) subjecting said reaction mixture to amplification conditions suitable to amplify at least a portion of said target region.

In another aspect, the invention provides a method for determining the presence of N. gonorrhoeae in a test sample. The method according to this aspect of the invention comprises (a) contacting a test sample with a composition comprising at least one primer pair comprising a forward primer and a reverse primer capable of hybridizing to a target region of the N. gonorrhoeae pilin E (pil E) gene consisting of SEQ ID NO:12 to form a reaction mixture; and (b) subjecting said reaction mixture to amplification conditions suitable to amplify at least a portion of said target region in another aspect, the invention provides a method for determining the presence of N. gonorrhoeae in a test sample, said method comprising the steps of (a) contacting a test sample with a composition comprising at least one primer pair comprising a forward primer and a reverse primer capable of hybridizing to a target region of the N. gonorrhoeae opa gene consisting of SEQ ID NO:2 and at least one primer pair comprising a forward primer and a reverse primer capable of hybridizing to a target region of the N. gonorrhoeae por A pseudo-gene consisting of SEQ ID NO:7 to form a reaction mixture; and (b) subjecting said reaction mixture to amplification conditions suitable to amplify at least a portion of said target region.

In another aspect, the invention provides a method for determining the presence of N. gonorrhoeae in a test sample, said method comprising the steps of (a) contacting a test sample with a composition comprising at least one primer pair comprising a forward primer and a reverse primer capable of hybridizing to a target region of the N. gonorrhoeae opa gene consisting of SEQ ID NO:2 and at least one primer pair comprising a forward primer and a reverse primer capable of hybridizing to a target region of the N. gonorrhoeae pil E gene consisting of SEQ ID NO:12 to form a reaction mixture; and (b) subjecting said reaction mixture to amplification conditions suitable to amplify at least a portion of said target region.

In another aspect, the invention provides a method for determining the presence of N. gonorrhoeae in a test sample, said method comprising the steps of (a) contacting a test sample with a composition comprising at least one primer pair comprising a forward primer and a reverse primer capable of hybridizing to a target region of the N. gonorrhoeae por A pseudo-gene consisting of SEQ ID NO:7 and at least one primer pair comprising a forward primer and a reverse primer capable of hybridizing to a target region of the N. gonorrhoeae pil E gene consisting of SEQ ID NO:12 to form a reaction mixture; and (b) subjecting said reaction mixture to amplification conditions suitable to amplify at least a portion of said target region.

In another aspect, the invention provides a method for determining the presence of N. gonorrhoeae in a test sample, said method comprising the steps of (a) contacting a test sample with a composition comprising at least one primer pair comprising a forward primer and a reverse primer capable of hybridizing to a target region of the N. gonorrhoeae opa gene consisting of SEQ ID NO:2, at least one primer pair comprising a forward primer and a reverse primer capable of hybridizing to a target region of the N. gonorrhoeae por A pseudo-gene consisting of SEQ ID NO:7, and at least one primer pair comprising a forward primer and a reverse primer capable of hybridizing to a target region of the N. gonorrhoeae pil E gene consisting of SEQ ID NO:12 to form a reaction mixture; and (b) subjecting said reaction mixture to amplification conditions suitable to amplify at least a portion of said target region.

In another aspect, the invention provides a set of oligonucleotides for use in amplifying a target region of nucleic acid derived from the opa gene of N. gonorrhoeae, the set of oligonucleotides comprising a forward primer and a reverse primer, each primer having a target binding region up to 30 nucleotides in length which contains at least 10 contiguous nucleotides that are perfectly complementary to an at least 10 contiguous nucleotide region present in a target sequence consisting of SEQ ID NO:2.

In another aspect, the invention provides a set of oligonucleotides for use in amplifying a target region of nucleic acid derived from the por A pseudo-gene of N. gonorrhoeae, the set of oligonucleotides comprising a forward primer and a reverse primer, each primer having a target binding region up to 30 nucleotides in length, which contains at least 10 contiguous nucleotides that are perfectly complementary to an at least 10 contiguous nucleotide region present in a target sequence consisting of SEQ ID NO:7.

In another aspect, the invention provides a set of oligonucleotides for use in amplifying a target region of nucleic acid derived from the pil E gene of N. gonorrhoeae, the set of oligonucleotides comprising a forward primer and a reverse primer, each primer having a target binding region up to 30 nucleotides in length that contains at least 10 contiguous nucleotides, which are perfectly complementary to an at least 10 contiguous nucleotide region present in a target sequence consisting of SEQ ID NO:12.

In another aspect, the invention provides an oligonucleotide for use in amplifying a target region of nucleic acid derived from N. gonorrhoeae, said oligonucleotide having a target binding region of up to 30 bases in length, which stably hybridizes to a target sequence selected from the group consisting of SEQ ID NO:2, SEQ ID NO:7, and SEQ ID NO:12.

In another aspect, the invention provides a kit for detecting the presence of N. gonorrhoeae in a test sample, in accordance with this aspect of the invention, the kit comprises (a) at least one oligonucleotide comprising a target binding region sequence selected from the group consisting of SEQ ID NO:3, SEQ ID NO:4, SEQ ID NO:8, SEQ ID NO:9, SEQ ID NO:13, and SEQ ID NO:14; (b) amplification reagents; and (c) written instructions describing amplification conditions suitable to detect the presence of N. gonorrhoeae in a test sample.

The invention thus provides methods, reagents, and kits for determining the presence of N. gonorrhoeae in a test sample.

### SEQUENCE LISTING

SEQ ID NO:1: N. gonorrhoeae opa gene full length (Genbank Ref. X52364, incorporated herein by reference)

SEQ ID NO:2: N. gonorrhoeae opa gene target region (nt 451-679 of SEQ ID NO:1)

SEQ ID NOS:3-5: primers and probes for opa assay

SEQ ID NO:6: N. gonorrhoeae por A pseudo-gene full length (Genbank Ref, AJ223447, incorporated herein by reference)

SEQ ID NO:7: N. gonorrhoeae por A pseudo-gene target region (nt 927-1003 of SEQ ID NO:6)

SEQ ID NOS:8-10: primers and probes for por A pseudo-gene assay

SEQ ID NO:11: N. gonorrhoeae pil E gene full length (Genbank Ref. X66830.1, incorporated herein by reference)

SEQ ID NO:12: N. gonorrhoeae pil E gene target region (nt 114-265 of SEQ ID NO:11)

SEQ ID NOS:13-15: primers and probes for pil E assay

### DETAILED DESCRIPTION

Unless specifically defined herein, all terms used herein have the same meaning as they would to one skilled in the art of the present invention. Practitioners are particularly directed to Sambrook et al. Molecular Cloning: A Laboratory Manual, 2d ed., Cold Spring Harbor Press, Plainsview, N.Y., 1989; and Ausubel et al, Current Protocols in Molecular Biology, John Wiley & Sons, New York, 1999, for definitions and terms of art.

The following definitions are provided in order to provide clarity with respect to the terms as they are used in the specification and claims to describe the present invention.

The term “specifically hybridize” as used herein refers to the ability of a nucleic acid to bind detectably and specifically to a second nucleic acid. Polynucleotides specifically hybridize with target nucleic acid strands under hybridization and wash conditions that minimize appreciable amounts of detectable binding to non-specific nucleic acids. Stringent conditions that can be used to achieve specific hybridization are known in the art.

A “target sequence” or “target nucleic acid sequence” as used herein means a nucleic acid sequence of N. gonorrhoeae, such as a target region of the opa gene (e.g., SEQ ID NO:2), or complement thereof, a target region of the por A pseudo-gene (e.g., SEQ ID NO:7), or complement thereof, or a target region of the pil E gene (e.g. SEQ ID NO:12), or complement thereof, that is amplified, detected, or both amplified and detected using one or more of the oligonucleotide primers provided herein. Additionally, while the term target sequence sometimes refers to a double stranded nucleic acid sequence, those skilled in the art will recognize that the target sequence can also be single stranded. In cases where the target is double stranded, polynucleotide primer sequences of the present invention preferably will amplify both strands of the target sequence. As described in Examples 1-3, the primer sequences of the present invention are selected for their ability to specifically hybridize with a range of different N. gonorrhoeae strains and to not hybridize to near neighbor organisms.

The term “test sample” as used herein refers to a sample taken from a subject or other source that is suspected of containing or potentially contains a N. gonorrhoeae target sequence. The test sample can be taken from any biological source, such as, for example, tissue, blood, saliva, sputa, mucus, sweat, urine, urethral swabs, cervical swabs, urogenital or anal swabs, conjunctival swabs, ocular lens fluid, cerebral spinal fluid, milk, ascites fluid, synovial fluid, peritoneal fluid, amniotic fluid, fermentation broths, cell cultures, chemical reaction mixtures and the like. The test sample can be used (i) directly as obtained from the source, or (ii) following a pre-treatment to modify the character of the sample. Thus, the test sample can be pre-treated prior to use, for example, by preparing plasma or serum from blood, disrupting cells or viral particles, preparing liquids from solid materials, diluting viscous fluids, filtering liquids, concentrating liquids, inactivating interfering components, adding reagents, purifying nucleic acids, and the like.

The term “label” as used herein means a molecule or moiety having a property or characteristic that is capable of detection and, optionally, of quantitation. A label can be directly detectable, as with, for example (and without limitation), radioisotopes, fluorophores, chemiluminophores, enzymes, colloidal particles, fluorescent microparticles and the like; or a label may be indirectly detectable as with, for example, specific binding members. It will be understood that directly detectable labels may require additional components such as, for example, substrates, triggering reagents, quenching moieties, light, and the like to enable detection and/or quantitation of the label. When indirectly detectable labels are used, they are typically used in combination with a “conjugate.” A conjugate is typically a specific binding member that has been attached or coupled to a directly detectable label. Coupling chemistries for synthesizing a conjugate are well known in the art and can include, for example, any chemical means and/or physical means that do not destroy the specific binding property of the specific binding member or the detectable property of the label. As used herein, “specific binding member” means a member of a binding pair, i.e., two different molecules where one of the molecules through, for example, chemical or physical means, specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors car substrates and enzymes; and the like,

A polynucleotide, in the context of the present invention, is as nucleic acid polymer of ribonucleic acid (RNA), deoxyribonucleic acid (DNA), modified RNA or DNA, or RNA or DNA mimetics (such as, without limitation, PNAs) and derivatives thereof, and homologues thereof. Thus, polynucleotides include polymers composed of naturally occurring nucleobases, sugars, and covalent intenucleoside (backbone) linkages as well as polymers having non-naturally-occurring portions that function similarly. Such modified or substituted nucleic acid polymers are well known in the art and for the purposes of the present invention, are referred to as “analogues.” For ease of preparation and familiarity to the skilled artisan, polynucleotides are preferably modified or unmodified polymers of deoxyribonucleic acid or ribonucleic acid.

As used herein, the term “primer” means a polynucleotide that can serve to initiate a nucleic acid chain extension reaction. Typically, primers have a length of 10 to about 50 nucleotides, although primers can be longer than 50 nucleotides.

As used herein, the term “sequence identity” or “percent identical” as applied to nucleic acid molecules is the percentage of nucleic acid residues in a candidate nucleic acid molecule sequence that are identical with a subject nucleic acid molecule sequence (such as the nucleic acid molecule sequence set forth in SEQ ID NO: 2), after aligning the sequences to achieve the maximum percent identity, and not considering any nucleic acid residue substitutions as part of the sequence identity. No gaps are introduced into the candidate nucleic acid sequence in order to achieve the best alignment. Nucleic acid sequence identity can be determined in the following manner. The subject polynucleotide molecule sequence is used to search a nucleic acid sequence database, such as the Genbank database, using the program BLASTN version 2.1 (based on Altschul et al., Nucleic Acids Research 25:3389-3402, 1997). The program is used in the ungapped mode. Default filtering is used to remove sequence homologies due to regions of low complexity as defined in J. C. Wootton and S. Federhen, Methods in Enzymology 266:554-571, 1996. The default parameters of BLASTN are utilized.

The present invention further encompasses homologues of the polynucleotides (i.e., primers and detection probes) having nucleic acid sequences set forth in SEQ ID NOS:3-5, 8-10, and 13-15. As used herein, the term “homologues” refers to nucleic acids having one or more alterations in the primary sequence set forth in any one of SEQ ID NOS:3-5, 8-10, and 13-15, that does not destroy the ability of the polynucleotide to specifically hybridize with a target sequence, as described above. Accordingly, a primary sequence can be altered, for example, by the insertion, addition, deletion or substitution of one or more of the nucleotides of, for example, SEQ ID NOS:3-5, 8-10, and 13-15. Thus, in one embodiment, homologues have a length in the range of from 10 to 30 nucleotides and have a consecutive sequence of at least 10, 11, 12, 13, 14, 15, 16, 17, 18, 19 20, 21, 22, 23, or more nucleotides of the nucleic acid sequences of SEQ ID NOS:3-5, 8-10, and 13-15 and will retain the ability to specifically hybridize with a target sequence, as described above. Ordinarily, the homologues will have a nucleic acid sequence having at least 85%, 90%, or 95% nucleic acid sequence identity with a nucleic acid sequence set forth in SEQ ID NOS:3-5, 8-10, and 13-15. In some embodiments, homologues have a length in the range of from 10 to 30 nucleotides and have a nucleotide sequence substantially identical to a nucleic acid sequence set forth as SEQ ID NOS:3-5, 8-10 and 13-15, with the difference being the presence of 1, 2 or 3 mismatches, provided that the homologues do not contain two or more consecutive mismatches.

The polynucleotides of the present invention thus comprise primers and probes that specifically hybridize to a target sequence of the invention—for example, the nucleic acid molecules having any one of the nucleic acid sequences set forth in SEQ ID NOS:3-5, 8-10, and 13-45, including analogues and/or derivatives of said nucleic acid sequences and homologues thereof, that can specifically hybridize with a target sequence of the invention. As described below, polynucleotides of the invention can be used as primers and/or probes to amplify or detect N. gonorrhoeae.

The polynucleotides according to the present invention can be prepared by conventional techniques well known to those skilled in the art. For example, the polynucleotides can be prepared using conventional solid-phase synthesis using commercially available equipment, such as that available from Applied Biosystems USA Inc. (Foster City, Calif.), DuPont, (Wilmington, Del.), or (Bedford, Mass.). Modified polynucleotides, such as phosphorothioates and alkylated derivatives, can also be readily prepared by similar methods known in the art. See, for example, U.S. Pat. Nos. 5,464,746; 5,424,414; and 4,948,882.

The polynucleotides according to the present invention can be employed directly as probes for the detection or quantitation, or both, of N. gonorrhoeae nucleic acids in a test sample.

In one aspect, the methods comprise detecting the presence of a target region of the opacity (opa) gene of N. gonorrhoeae in a test sample. The opacity gene is encoded by at least 11 intact structural genes, more than one of which can be expressed at one time (T. D. Connell, et al, Mol. Microbiol. 4:439 449, 1990). The gene has near perfect identity over approximately 80% of the length of the coding sequence for mature opa protein, suggesting that despite antigenic variation, portions of the gene sequence are stable (J. Dempsey, et al., J, Bacteriol, 173:5476-5486, 1991; K. S. Bhatt, et al, Molec. Microbiol, 518:1889-1901, 1991). The presence of multiple copies results in a duplication of sequence within the genome that may increase the sensitivity of assays designed targeting this gene. The full-length nucleotide sequence of the opa gene of N. gonorrhoeae from the reference opa gene of N. gonorrhoeae sequence (Genbank Ref. X52634) is set forth as SEQ ID NO:1. In one embodiment, the target region consists of SEQ ID NO:2 (nucleotides 451 to 679 of SEQ ID NO:11.

In accordance with one embodiment of the invention, the method comprises contacting a test sample with a composition comprising at least one primer pair comprising a forward primer and a reverse primer capable of hybridizing to a target region of the opa gene of N. gonorrhoeae consisting of SEQ ID NO:2 to form a reaction mixture and subjecting said reaction mixture to amplification conditions suitable to amplify the target region. The amplification conditions are suitable to allow hybridization between the target sequence and the primer pair. In one embodiment, the composition comprises a primer having a target-binding region consisting of SEQ ID NO:3. In one embodiment, the composition comprises a primer having a target-binding region consisting of SEQ ID NO:4. In some embodiments, the amplified target region is then detected by a probe that hybridizes to the amplified target region using methods well known in the art. In one embodiment, the probe comprises a target-binding region consisting of SEQ ID NO:5.

In one aspect, the methods comprise detecting the presence of a target region of the porin A (por A) pseudo-gene of N. gonorrhoeae in a test sample. The porin gene is present only in the Neisseria gonorrhoeae and Neisseria meningitis members of the Neisseria species. In Neisseria gonorrhoeae, the porA gene is not expressed due to a frame-shift and promoter mutations resulting in a pseudo-gene that is not under selective pressure from the immune system (LP. Derrick, et al., Inf. and Immunity 67:2406-2413, 1999), The por A pseudo-gene is highly conserved across Neisseria gonorrhoeae subtypes (M. Unemo, et al, APMIS 113:410-419, 2005); D. Whiley, et al., Pathology 38:445-448, 2006). The full-length nucleotide sequence of the por A pseudo-gene of N. gonorrhoeae from the reference por A pseudo-gene of N. gonorrhoeae sequence (Genbank Ref. 10223447) is set forth as SEQ ID NO:6. In one embodiment, the target region consists of SEQ ID NO:7 (nucleotides 927 to 1003 of SEQ ID NO:6).

In accordance with one embodiment of the invention, the method comprises contacting a test sample with a composition comprising at least one primer pair comprising a forward primer and a reverse primer capable of hybridizing to a target region of the por A pseudo-gene of N. gonorrhoeae consisting of SEQ ID NO:7 to form a reaction mixture and subjecting said reaction mixture to amplification conditions suitable to amplify the target region. The amplification conditions are suitable to allow hybridization between the target sequence and the primer pair. In one embodiment, the composition comprises a primer having a target-binding region consisting of SEQ ID NO:8, in one embodiment, the composition comprises a primer having a target-binding region consisting of SEQ ID NO:9, in some embodiments, the amplified target region is then detected by a probe that hybridizes to the amplified target region using methods well known in the art. In one embodiment, the probe comprises a target-binding region consisting of SEQ ID NO:10.

In one aspect, the methods comprise detecting the presence of a target region of the pil E gene of N. gonorrhoeae in a test sample. The full length nucleotide sequence of the pil E gene of N. gonorrhoeae from the reference pil E gene of N. gonorrhoeae sequence (Genbank Ref. X66830.1) is set forth as SEQ ID NO:11. In one embodiment, the target region consists of SEQ ID NO:12 (nucleotides 114 to 265 of SEQ ID NO:11), in accordance with one embodiment of the invention, the method comprises contacting a test sample with a composition comprising at least one primer pair comprising a forward primer and a reverse primer capable of hybridizing to a target region of the pil E gene of N. gonorrhoeae consisting of SEQ ID NO:12 to form a reaction mixture and subjecting said reaction mixture to amplification conditions suitable to amplify the target region. The amplification conditions are suitable to allow hybridization between the target sequence and the primer pair. In one embodiment, the composition comprises a primer having a target-binding region consisting of SEQ ID NO:13. In one embodiment, the composition comprises a primer having a target-binding region consisting of SEQ ID NO:14. In some embodiments, the amplified target region is then detected by a probe that hybridizes to the amplified target region using methods well known in the art. In one embodiment, the probe comprises a target-binding region consisting of SEQ ID NO:15.

The polynucleotides (i.e. primers and probes) of the present invention may incorporate one or more detectable labels. Detectable labels are molecules or moieties having a property or characteristic that can be detected directly or indirectly and are chosen such that the ability of the polynucleotide to hybridize with its target sequence is not adversely affected. Methods of labeling nucleic acid sequences are well known in the art (see, for example, Ausubel et al., Current Protocols in Molecular Biology, Wiley & Sons, New York, 1997 and updates).

Amplification procedures are well-known in the art and include, but are not limited to, polymerase chain reaction (PCR), TMA, rolling circle amplification, nucleic acid sequence based amplification (NASBA), and strand displacement amplification (SDA). One skilled in the art will understand that for use in certain amplification techniques the primers may need to be modified, for example, for SDA the primer comprises additional nucleotides near its 5′ end that constitute a recognition site for a restriction endonuclease. Similarly, for NASBA the primer comprises additional nucleotides near the 5′ end that constitute an RNA polymerase promoter. Polynucleotides thus modified are considered to be within the scope of the present invention.

Certain criteria are taken into consideration when selecting the primers and probes for use in the methods of the invention. For example, for primer pairs for use in the amplification reactions, the primers are selected such that the likelihood of forming 3′ duplexes is minimized, and such that the melting temperatures (Tm) are sufficiently similar to optimize annealing to the target sequence and minimize the amount of non-specific annealing. In this context, the polynucleotides according to the present invention are provided in combinations that can be used as primers in amplification reactions to specifically amplify target nucleic acid sequences.

The amplification method of the present invention generally comprises (a) forming a reaction mixture comprising nucleic acid amplification reagents, at least one set of primers of the present invention, and a test sample suspected of containing a at least one target sequence, and (b) subjecting the mixture to amplification conditions to generate at least one copy of a nucleic acid sequence complementary to the target sequence.

Step (b) of the above methods can be repeated any suitable number of times (prior to detection of the amplified region), e.g., by thermal cycling the reaction mixture between 10 and 100 times, typically between about 20 and about 60 times, more typically between about 25 and about 45 times, such as between about 30 and 40 times.

Nucleic acid amplification reagents include reagents that are well known and may include, but are not limited to, an enzyme having at least polymerase activity, enzyme cofactors such as magnesium or manganese; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate.

Amplification conditions are conditions that generally promote annealing and extension of one or more nucleic acid sequences. It is well known that such annealing is dependent in a rather predictable manner on several parameters, including temperature, ionic strength, sequence length, complementarity, and G:C content of the sequences. For example, lowering the temperature in the environment of complementary nucleic acid sequences promotes annealing. For any given set of sequences, melt temperature (or Tm) can be estimated by any of several known methods. Typically, diagnostic applications utilize hybridization temperatures that are about 10° C. (e.g., 2° C. to 1.8″ C.) below the melt temperature. Ionic strength or “salt” concentration also impacts the melt temperature, since small cations tend to stabilize the formation of duplexes by negating the negative charge on the phosphodiester backbone. Typical salt concentrations depend on the nature and valency of the cation, but are readily understood by those skilled in the art. Similarly, high G:C content and increased sequence length are also known to stabilize duplex formation, because G:C pairings involve 3 hydrogen bonds where A:T pairs have just two, and because longer sequences have more hydrogen bonds holding the sequences together. Thus, a high G:C content and longer sequence lengths impact the hybridization conditions by elevating the melt temperature.

Specific amplicons produced by amplification of target nucleic acid sequences using the polynucleotides of the present invention, as described above, can be detected by a variety of methods known in the art. For example, one or more of the primers used in the amplification reactions may be labeled such that an amplicon can be directly detected by conventional techniques subsequent to the amplification reaction. Alternatively, a probe consisting of a labeled version of one of the primers used in the amplification reaction, or a third polynucleotide distinct from the primer sequences that has been labeled and is complementary to a region of the amplified sequence can be added after the amplification reaction is complete. The mixture is then submitted to appropriate hybridization and wash conditions and the label is detected by conventional methods.

The amplification product produced as above can be detected during or subsequently to the amplification of the target sequence. Methods for detecting the amplification of a target sequence during amplification are outlined above, and described, for example, in U.S. Pat. No. 5,210,015. Gel electrophoresis can be employed to detect the products of an amplification reaction after its completion. Alternatively, amplification products are hybridized to probes, then separated from other reaction components, and detected using microparticles and labeled probes.

It will be readily appreciated that a procedure that allows both amplification and detection of target nucleic acid sequences to take place concurrently in a single unopened reaction vessel would be advantageous. Such a procedure would avoid the risk of “carry-over” contamination in the post-amplification processing steps, and would also facilitate high-throughput screening or assays and the adaptation of the procedure to automation. Furthermore, this type of procedure allows “real-time” monitoring of the amplification reaction as well as more conventional “end-point” monitoring.

The present invention thus includes the use of the polynucleotides in a method to specifically amplify and detect target nucleic acid sequences in a test sample in a single tube format. This may be achieved, for example, by including in the reaction vessel an intercalating dye such as SYBR Green or an antibody that specifically detects the amplified nucleic acid sequence. Alternatively, a third polynucleotide distinct from the primer sequences, which is complementary to a region of the amplified sequence, may be included in the reaction, as when a primer/probe set of the invention is used.

For use in an assay as described above, in which both amplification with polynucleotide primers and detection of target sequences using a polynucleotide probe occur concurrently in a single unopened reaction vessel, the polynucleotide probe preferably possesses certain properties. For example, since the probe will be present during the amplification reaction, it should not interfere with the progress of this reaction, and should also be stable under the reaction conditions. In addition, for real-time monitoring of reactions, the probe should be capable of binding its target sequence under the conditions of the amplification reaction, and to emit a signal only upon binding this target sequence. Examples of probe molecules that are particularly well suited to this type of procedure include molecular beacon probes and probes comprising a fluorophore covalently attached to the 5′ end of the probe and a quencher at the 3′ end (e.g., TaqMan® probes).

The present invention, therefore, contemplates the use of the polynucleotides as TaqMan® probes. As is known in the art, TaqMan® probes are dual-labeled fluorogenic nucleic acid probes composed of a polynucleotide complementary to the target sequence that is labeled at the 5′ terminus with a fluorophore and at the 3′ terminus with a quencher, TaqMan® probes are typically used as real-time probes in amplification reactions, in the free probe, the close proximity of the fluorophore and the quencher ensures that the fluorophore is internally quenched, During the extension phase of the amplification reaction, the probe is hound to the target sequence, cleaved by the 5′ nuclease activity of the polymerase and the fluorophore is released. The released fluorophore can then fluoresce and thus produces a detectable signal.

Suitable fluorophores and quenchers for use with the polynucleotides of the present invention can be readily determined by one skilled in the art (see also Tyagi et al., Nature Biotechnol. 16:49-53, 1998; Marras et al., Genet. Anal.: Biomolec. Eng. 14:151-156, 1999), Many fluorophores and quenchers are available commercially, for example, from Molecular Probes (Eugene, Oreg.) or Biosearch Technologies, Inc. (Novato, Calif.). Examples of fluorophores that can be used in the present invention include, but are not limited to, fluorescein and fluorescein derivatives such as carboxy fluorescein (FAM®), a dihalo-(C1 to C8)dialkoxycarboxyfluorescein, 5-(2′-aminoethyl)aminonaphthalene-1-sulphonic acid (EDANS), coumarin and coumarin derivatives, Lucifer yellow, Texas red, tetramethylrhodamine, tetrachloro-6-carboxyfluoroscein, 5-carboxyrhodamine, cyanine dyes and the like, Quenchers include, but are not limited to, DABCYL, 4′-(4-dimethylaminophenylazo)benzoic acid (DABSYL), 4dimethylaminophenylazophenyl-4-dimethylaminophenyl azophenyl-4′-maleimide (DABMI), tetramethylrhodamine, carboxytetramethylrhodamine (TAMRA), dihydrocyclopyrroloindole tripeptide minor groove binder (MGB®) dyes and the like. Methods of coupling fluorophores and quenchers to nucleic acids are well known in the art. The present invention thus includes the use of the polynucleotides in a method to specifically amplify and detect target nucleic acid sequences in a test sample in a single tube format. This may be achieved, for example, by including in the reaction vessel an intercalating dye such as SYBR Green or an antibody that specifically detects the amplified nucleic acid sequence. Alternatively, a third polynucleotide distinct from the primer sequences, which is complementary to a region of the amplified sequence, may be included in the reaction, as when a primer/probe set of the invention is used.

In accordance with the present invention, therefore, the combinations of two primers and at least one probe, as described above, can be used in either end-point amplification and detection assays, in which the strength of the detectable signal is measured at the conclusion of the amplification reaction, or in real-time amplification and detection assays, in which the strength of the detectable signal is monitored throughout the course of the amplification reaction.

The polynucleotides according to the present invention can also be used in assays to detect the presence and/or quantitate the amount of N. gonorrhoeae nucleic acid present in a test sample. Thus, the polynucleotides according to the present invention can be used in a method to specifically amplify, detect, and quantitate target nucleic acid sequences in a test sample, which generally comprises the steps of (a) forming a reaction mixture comprising nucleic acid amplification reagents, at least one polynucleotide probe sequence that incorporates a label that produces a detectable signal upon hybridization of the probe to its target sequence, at least one polynucleotide primer, and a test sample that contains one or more target nucleic acid sequences; (b) subjecting the mixture to amplification conditions to generate at least one copy of the target nucleic acid sequence, or a nucleic acid sequence complementary to the target sequence; (c) hybridizing the probe to the target nucleic acid sequence or the nucleic acid sequence complementary to the target sequence, so as to form a probe:target hybrid; (d) detecting the probe:target hybrid by detecting the signal produced by the hybridized labeled probe; and (e) comparing the amount of the signal produced to a standard as an indication of the amount of target nucleic acid sequence present in the test sample.

One skilled in the art will understand that, as outlined above, step (b) of the above method can be repeated several times prior to step (c) by thermal cycling the reaction mixture by standard techniques known in the art.

Various types of standards for quantitative assays are known in the art. For example, the standard can consist of a standard curve compiled by amplification and detection of known quantities of N. gonorrhoeae nucleic acids under the assay conditions. Alternatively, an internal, standard can be included in the reaction. Such internal standards generally comprise a control target nucleic acid sequence and a control polynucleotide probe. The internal standard can optionally further include an additional pair of primers. The primary sequence of these control primers may be unrelated to the polynucleotides of the present invention and specific for the control target nucleic acid sequence.

In another aspect, the invention provides a set of oligonucleotides for use in amplifying a target region of nucleic acid derived from the opa gene of N. gonorrhoeae, the set of oligonucleotides comprising a forward primer and a reverse primer, each primer having a target binding region. In some embodiments, the target binding region is located at the 3′ end of the oligonucleotide. In some embodiments, the target binding region is from 10 to 30 nucleotides in length and contains at least 10 contiguous nucleotides that are perfectly complementary to an at least 10 contiguous nucleotide region present in a target sequence consisting of SEQ ID NO:2. In one embodiment, the forward primer comprises a target-binding region consisting of SEQ ID NO:3. In one embodiment, the reverse primer comprises a target-binding region consisting of SEQ ED NO:4. In one embodiment, the detection probe comprises a target-binding region consisting of SEQ ID NO:5.

In another aspect, the invention provides a set of oligonucleotides for use in amplifying a target region of nucleic acid derived from the porA pseudo-gene of N. gonorrhoeae, the set of oligonucleotides comprising a forward primer and a reverse primer, each primer having a target binding region. In some embodiments, the target binding region is located at the 3′ end of the oligonucleotide. In some embodiments, the target binding region is from 10 to 30 nucleotides in length, and contains at least 10 contiguous nucleotides that are perfectly complementary to an at least 10 contiguous nucleotide region present in a target sequence consisting of SEQ ID NO:7. in one embodiment, the forward primer comprises a target-binding region consisting of SEQ ID NO:8. In one embodiment, the reverse primer comprises a target-binding region consisting of SEQ ID NO:9. In one embodiment, the detection probe comprises a target-binding region consisting of SEQ ID NO:10.

In another aspect, the invention provides a set of oligonucleotides for use in amplifying a target region of nucleic acid derived from the pil E gene of N. gonorrhoeae, the set of oligonucleotides comprising a forward primer and a reverse primer, each primer having a target binding region. In some embodiments, the target binding region is located at the 3′ end of the oligonucleotide. In some embodiments, the target binding region is from 10 to 30 nucleotides in length, and contains at least 10 contiguous nucleotides that are perfectly complementary to an at least 10 contiguous nucleotide region present in a target sequence consisting of SEQ ID NO:12. In one embodiment, the forward primer comprises a target-binding region consisting of SEQ ID NO:13. In one embodiment, the reverse primer comprises a target-binding region consisting of SEQ ID NO:14. In one embodiment, the detection probe comprises a target-binding region consisting of SEQ ID NO:15.

In another aspect, the invention provides an oligonucleotide for use in amplifying a target region of nucleic acid derived from N. gonorrhoeae, said oligonucleotide having a target binding region. In some embodiments, the target binding region is located at the 3′ end of the oligonucleotide. In some embodiments, the target binding region is from 10 to 30 bases in length, and stably hybridizes to a target sequence selected from the group consisting of SEQ ID NO:2, SEQ ID NO:7, and SEQ ID NO:12. In one embodiment, the oligonucleotide comprises a target-binding region that contains at least 10 contiguous nucleotides that are perfectly complementary to at least 10 contiguous nucleotides in said target sequence.

In another aspect, the invention provides a kit for determining the presence of N. gonorrhoeae in a test sample. In accordance with this aspect of the invention, the kit comprises (a) at least one oligonucleotide comprising a target binding region sequence selected from the group consisting of SEQ ID NO:3, SEQ ID NO:4, SEQ ID NO:5, SEQ ID NO:8, SEQ ID NO:9, SEQ ID NO:10, SEQ ID NO:13 SEQ ID NO:14, and SEQ ID NO:15; (b) amplification reagents; and (c) written instructions describing amplification conditions suitable to detect the presence of N. gonorrhoeae in a test sample.

In one embodiment, kits for the detection of N. gonorrhoeae nucleic acids may additionally contain a control target nucleic acid and a control polynucleotide probe. Thus, in one embodiment of the present invention, the kits comprise one of the above combinations of polynucleotides comprising at least two primers and at least one probe, together with a control target nucleic acid sequence, which can be amplified by the specified primer pair, and a control polynucleotide probe. The present invention further provides kits that include control primers, which specifically amplify the control target nucleic acid sequence.

The kits can optionally include amplification reagents, reaction components, and/or reaction vessels. Typically, at least one sequence bears a label, but detection is possible without this. Thus, one or more of the polynucleotides provided in the kit may have a detectable label incorporated, or the kit may include reagents for labeling the polynucleotides. One or more of the components of the kit may be lyophilized and the kit may further comprise reagents suitable for the reconstitution of the lyophilized components.

The polynucleotides, methods, and kits of the present invention are useful in clinical or research settings for the detection and/or quantitation of N. gonorrhoeae nucleic acids. Thus, in these settings, the polynucleotides can be used in assays to diagnose N. gonorrhoeae infection in a subject, or to monitor the quantity of a N. gonorrhoeae target nucleic acid sequence in a subject infected with N. gonorrhoeae.

The following examples merely illustrate the best mode now contemplated for practicing the invention, but should not be construed to limit the invention.

### EXAMPLES

### Example 1

This example describes the rationale for selection of the target region of the opa gene of N. gonorrhoeae to be used in a diagnostic test.

Methods:

The target region was selected based on conserved regions of the opa gene of N. gonorrhoeae that were non-homologous with Neisseria meningitis following construction of an alignment incorporating sequences of the opa gene of fifteen N. gonorrhoeae strains and two N. meningitis strains. Assays were analyzed in silica by using BLASTN to avoid cross-reactivity to commensal organisms. Sequences having greater than 50% homology to any sequence that was non-gonococcal were discarded. Selected sequences were then tested on multiple N. gonorrhoeae strains from diverse geographical locations. Criteria for selecting a primer and probe set were the ability to detect all gonococcal strains tested, including variants such as the C4bp, a strain that leads to asymptomatic disease. In addition, primers and probes were tested for specificity by challenging with large amount of genomic DNA from near neighbor organisms and organisms found in the same biological compartment. Only primer and probe sets showing no cross-reactivity were selected. All tests confirmed the assay as both sensitive and specific.

### Example 2

This example describes the rationale for selection of the target region of the por A pseudo-gene of N. gonorrhoeae to be used in a diagnostic test.

Methods:

The target region was selected based on an alignment incorporating sequences of the por A pseudo-gene of seven N. gonorrhoeae strains and forty-three N. meningitis strains.

Assays were analyzed in silico by using BLASTN to avoid cross-reactivity to commensal organisms. Sequences having greater than 50% homology to any sequence that was non-gonococcal were discarded. Selected sequences were then tested on multiple N. gonorrhoeae strains from diverse geographical locations. Criteria for selecting a primer and probe set were the ability to detect all gonococcal strains tested, including variants such as the C4bp, a strain that leads to asymptomatic disease. In addition, primers and probes were tested for specificity by challenging with large amount of genomic DNA from near neighbor organisms and organisms found in the same biological compartment. Only primer and probe sets showing no cross-reactivity were selected. All tests confirmed the assay as both sensitive and specific.

### Example 3

This example describes the rationale for selection of the target region of the pil E gene of N. gonorrhoeae to be used in a diagnostic test.

Methods:

The target region was selected based on an alignment incorporating sequences of the pil E gene of thirty-eight N. gonorrhoeae strains and two N. meningitis strains. Assay designs were tested in silico using BLAST to minimize cross-reactivity with other organisms. The assay's were tested with eleven geographically diverse N. gonorrhoeae strains and non-physiologically high amount of N. meningitis genomic DNA. The assay was able to differentiate between the two organisms with a differential of ten cycles. The assay was further challenged with an additional fifteen N. gonorrhoeae strains. The initial assay successfully detected these additional strains but failed to detect a strain of N. gonorrhoeae that has differential binding to c4bp, associated with less symptomatic disease. The assay was modified such that these N. gonorrhoeae variants were successfully detected.

### Example 4

In this example, real-time polymerase chain reaction (rt-PCR) assays were performed on clinical samples that were known to be positive or negative for Neisseria gonorrhoeae (gonococcal).

Methods:

Gonococcal (n=76) and non-gonococcal (n=36) isolates were obtained from the Neisseria Reference Laboratory (NRL) of the University of Washington, Seattle, Wash. Gonococcal isolates were selected based on geographic and anatomic site diversity. Included were isolates from the United States and seven other countries. Within the United States, isolates from nine different cities were included. Anatomic site diversity included gonococci recovered from the urethra, cervix, rectum, pharynx, fallopian tubes, blood, and synovial fluid. Further, five gonococcal isolates of a type with known mutations in the opa gene were included. Additionally, isolates (37) of 16 other bacterial species, including N. cinerea, N. sicca, N. lactamica, N meningitidis and Branhamella catarrhalis were assayed using primers and probes targeted to the opa gene and the por A pseudo-gene of N. gonorrhoeae. All tests were performed without knowledge of the identity of the bacterial isolates. The gonococcal strain ATCC 49226 was used as a control. Results from clinical sample testing are summarized in Table 4. In addition, the opa, por A, and pil E assays described herein were further validated in a set of 400 clinical samples from commercial sex workers form Mombasa, Kenya. Test results were compared to GenProbe APTIMA COMBO 2 assay test results, gonococcal smear, and gonococcal culture and showed excellent concordance (data not shown). Specificity testing results from near neighbor organisms are summarized in Table 5.

The details of each assay are described below.

A. Target: N. gonorrhoeae opa gene target region
(SEQ ID NO: 2)

Primers:
Forward Primer
(SEQ ID NO: 3)
5′-ATAAGGAGCCGAAAATGAATCCAGC-3′
Reverse Primer
(SEQ ID NO: 4)
5′-GTTTCTGAAATAATCGCTTACCGT-3′
Probe:
(SEQ ID NO: 5)
5′-FAM-ATGTGCAGGCGGATT-MGB-3′

The primers, probe, and reaction master mix were obtained from Applied. Biosystems (ABI) of Carlsbad, Calif.,

The components of the reaction mixture used in the rt-PCR assay are set forth in Table 1.

TABLE 1

| | | |
|---|---|---|
| Component | /single reaction | Final conc./reaction |

| | | | |
|---|---|---|---|
| ABI 2X mastermix* | 12.5 | μl | |
| Forward primer (20 μM) | 0.5 | μl | 400 nM (20 pMol) |
| Reverse primer (20 μM) | 0.5 | μl | 400 nM (20 pMol) |
| Probe (100 μM) | 0.025 | μl | 200 nM (10 pMol) |
| Template (10 ng gDNA) | 2.5 | μl | |
| PCR grade H₂O | 8.975 | μl | |
| Total volume | 25 | μl | |

*(contains dNTPs, Taq DNA polymerase, MgCl₂ and reaction buffer)

The rt-PCR reaction was performed using an ABI 7:300 Realtime PCR system under the following conditions:

The Detector on the instrument was set to “FAM (no quench)”
Initial 50° C., 2-minute incubation for UNG nuclease digestion (optional)

| | | |
|---|---|---|
| Initial denaturation at | 95° C., 10 minutes | |
| | 40 cycles of Denaturation | 95° C., 15 sec |
| | Anneal/Extension | 60° C., 1 min |

Data was collected in the 60° C. anneal/extension step.

Results

The mean cycle-threshold (Ct) values obtained, using the opa gene as a target, from the gonococcal-positive samples ranged from 16.3 to 22.6 and strains with known opa mutations yielded indistinguishable values from other gonococci (P>0.4). Among 36 isolates of other bacterial species, 22 were undetectable (Ct>40) and the remaining yielded mean Ct values of 34.9-40.0. Within each panel, Ct values for gonococci exceeded Ct values for other species by 13.8. Values of the control strain, ATCC 49226, were highly correlated (R=0.96, Spearman's Rho) between test panels,

B. Target: N. gonorrhoeae por A gene target region
(SEQ ID NO: 7)

Primers:
Forward Primer
(SEQ ID NO: 8)
5′-GGGCATGATGCTTTCTTTTTG-3′
Reverse Primer
(SEQ ID NO: 9)
5′-CGGTGTACCTGATGGTTTTTCA-3′
Probe:
(SEQ ID NO: 10)
5′-FAM-CGAGTGATACCGATCCA-MGB-3′

The primers, probe, and reaction master mix were obtained from Applied Biosystems (ABI) of Carlsbad, Calif.

The components of the reaction mixture used in the rt-PCR assay are set forth in Table 2,

TABLE 2

| | | |
|---|---|---|
| Component | /single reaction | Final conc./reaction |

| | | | |
|---|---|---|---|
| ABI 2X mastermix* | 12.5 | μl | |
| Forward primer (20 μM) | 1.0 | μl | 800 nM |
| Reverse primer (20 μM) | 1.0 | μl | 800 nM |
| Probe (100 μM) | 0.025 | μl | 200 nM |
| Template (10 ng gDNA) | 2.5 | μl | |
| PCR grade H₂O | 7.975 | μl | |
| Total volume | 25 | μl | |

*(contains dNTPS, Taq DNA polymerase, MgCl₂ and reaction buffer)

The rT-PCR reaction was performed using and ABI Realtime PCR system under the following conditions:

The Detector on the instrument was set to “FAM (no quench)”
Initial 50° C., 2 minutes incubation for UNG nuclease digestion (optional)

| | | |
|---|---|---|
| Initial denaturation at | 95° C., 10 minutes | |
| | 40 cycles of Denaturation | 95° C., 15 sec |
| | Anneal/Extension | 60° C., 1 min |

Data was collected in the 60° C. anneal/extension step.

Results

The mean cycle-threshold (Ct) values obtained, using the por A pseudo-gene as a target, from the gonococcal-positive samples ranged from 18.2 to 24.8. Other species yielded mean Ct values from 34.5 to 40; 20 were undetectable (Ct>40). The mean value of detectable isolates was 37.2. Within each panel, Ct values for gonococci exceeded Ct values for other species by 14.2. Likewise, values of the control strain, ATCC 49226, were highly correlated (R=0.94, Spearman's Rho) between test panels. Among gonococcal isolates, results of the opa and per A assays were correlated (R=0.74, Spearman's Rho).

C. Target: N. gonorrhoeae pil E gene target region
(SEQ ID NO: 12)

Primers:
Forward Primer
(SEQ ID NO: 13)
5′-GSAAGTTTCCGAAGCCATCCT-3′
Reverse Primer
(SEQ ID NO: 14)
5′-YAACBYYYTBAACATATTTGCCTTTGAT-3′
Probe:
(SEQ ID NO: 15)
5′-6FAM-ACGGCRHATGGCCSRVMVAVAACVVYKCCG-MGBNFQ-3′

The Standard Mixed Base symbols in the sequences above, and corresponding nucleotides, are shown below.

| Symbol | Nucleotide | Symbol | Nucleotide | Symbol | Nucleotide |
|---|---|---|---|---|---|
| R | A, G | S | C, G | V | A, C, G |
| Y | C, T | W | A, T | D | A, G, T |
| M | A, C | H | A, C, T | N | A, C, G, T |
| K | G, T | B | C, G, T | | |

The components of the reaction mixture used in the rt-PCR assay are set forth in Table 3.

TABLE 3

| | | |
|---|---|---|
| Component | /single reaction | Final conc./reaction |

| | | | |
|---|---|---|---|
| ABI 2X mastermix* | 12.5 | μl | 1X |
| Forward primer (20 μM) | 1.0 | μl | 800 nM |
| Reverse primer (20 μM) | 1.0 | μl | 800 nM |
| Probe (100 μM) | 0.2 | μl | 800 nM |
| Template (10 ng gDNA) | 1.0 | μl | |
| PCR grade H₂O | 9.3 | μl | |
| Total volume | 25 | μl | |

*(contains dNTPS, Taq DNA polymerase, MgCl₂ and reaction buffer)

The rT-PCR reaction was performed using an ABI 7300 Realtime PCR system under the following conditions:

The Detector on the instrument was set to “FAM (no quench)”
Initial 50° C., 2-minute incubation for UNG nuclease digestion (optional)

| | | |
|---|---|---|
| Initial denaturation at | 95° C., 10 mins | |
| | 40 cycles of Denaturation | 95° C., 15 sec |
| | Anneal/Extension | 60° C., 1 min |

Data was collected in the 60° C. anneal/extension step.

Results

The cycle-threshold (Ct) values obtained, using the pil E gene as a target, from the gonococcal strains and isolates tested ranged from 16-25, and were separated by a greater than ten Ct difference for non-gonococcal organisms.

Summary of Results

TABLE 4
CLINICAL SAMPLES TESTED

| | Geographic | Reason for | | opa | porA | pilE |
|---|---|---|---|---|---|---|
| NRL ID | Source | Inclusion | Description | Ct | Ct | Ct |
| C5 | US Pacific | Anatomic & | Isolate from | 17.51 | 20.21 | NT |
| | Northwest | clinical diversity | patient with | | | |
| | | | disseminated | | | |
| | | | gonococcal | | | |
| | | | infection | | | |
| C11 | US Pacific | Anatomic & | Rectal isolate | 18.09 | 20.48 | NT |
| | Northwest | clinical diversity | | | | |
| C14 | US Pacific | Anatomic & | Pharyngeal | 18.01 | 20.09 | NT |
| | Northwest | clinical diversity | isolate; tetM | | | |
| | | | determinate | | | |
| C21 | US Pacific | Anatomic & | Rectal | 18.38 | 21.07 | NT |
| | Northwest | clinical diversity | isolate; | | | |
| | | | ciprofloxacin | | | |
| | | | resistance | | | |
| C31 | US Pacific | Anatomic & | Pharyngeal | 17.20 | 19.95 | NT |
| | Northwest | clinical diversity | isolate; | | | |
| | | | ciprofloxacin | | | |
| | | | resistance | | | |
| C34 | US Pacific | Anatomic & | Rectal | 18.10 | 20.34 | NT |
| | Northwest | clinical diversity | isolate; tetM | | | |
| | | | determinate | | | |
| C42 | US Pacific | Anatomic & | Pharyngeal | 18.61 | 20.76 | NT |
| | Northwest | clinical diversity | isolate; | | | |
| | | | azithromycin | | | |
| | | | resistance | | | |
| C43 | US Pacific | Anatomic & | Rectal | 18.45 | 20.99 | NT |
| | Northwest | clinical diversity | isolate; | | | |
| | | | azithromycin | | | |
| | | | resistance | | | |
| C52 | US Pacific | Anatomic & | Pharyngeal | 17.36 | 20.95 | NT |
| | Northwest | clinical diversity | isolate | | | |
| C54 | US Pacific | Anatomic & | Pharyngeal | 17.18 | 20.54 | NT |
| | Northwest | clinical diversity | isolate | | | |
| C56 | US Pacific | Anatomic & | Isolate from | 17.99 | 21.45 | NT |
| | Northwest | clinical diversity | patient with | | | |
| | | | disseminated | | | |
| | | | gonococcal | | | |
| | | | infection; | | | |
| | | | ciprofloxacin | | | |
| | | | resistance | | | |
| C61 | US Pacific | Anatomic & | Pharyngeal | 16.91 | 20.74 | NT |
| | Northwest | clinical diversity | isolate | | | |
| C64 | US Pacific | Anatomic & | Rectal isolate | 17.61 | 21.15 | NT |
| | Northwest | clinical diversity | | | | |
| C76 | US Pacific | Anatomic & | Rectal | 17.15 | 20.45 | NT |
| | Northwest | clinical diversity | isolate; | | | |
| | | | ciprofloxacin | | | |
| | | | resistance | | | |
| 31426 | Seattle, WA | Anatomic & | Rectal isolate | 21.35 | 23.31 | 26.225 |
| | | clinical diversity | | | | |
| 38794 | Seattle, WA | Anatomic & | Salpingitis-- | 17.76 | 19.18 | 22.68 |
| | | clinical diversity | specimen | | | |
| | | | from | | | |
| | | | fallopian | | | |
| | | | tube | | | |
| 38885 | Seattle, WA | Anatomic & | Salpingitis-- | 16.38 | 18.69 | 20.59 |
| | | clinical diversity | specimen | | | |
| | | | from | | | |
| | | | fallopian | | | |
| | | | tube | | | |
| 39260 | Seattle, WA | Anatomic & | Salpingitis-- | 18.06 | 19.67 | 19.40 |
| | | clinical diversity | specimen | | | |
| | | | from the cds | | | |
| CAM | Cambodia | Geographic | Cervical | 17.76 | 19.50 | NT |
| 1211 | | diversity | isolate; | | | |
| | | | intermediate | | | |
| | | | resistance to | | | |
| | | | ciprofloxacin | | | |
| CAM | Cambodia | Geographic | Cervical | 17.27 | 19.13 | NT |
| 1250 | | diversity | isolate; β- | | | |
| | | | lactamase | | | |
| | | | positive | | | |
| DAL | Dallas, TX | Geographic | Urethral | 21.35 | 24.43 | 35.54 |
| 200708-3 | | diversity | isolate | | | |
| DAL | Dallas, TX | Geographic | Urethral | 20.10 | 21.96 | 32.59 |
| 200708-7 | | diversity | isolate | | | |
| DAL | Dallas, TX | Geographic | Urethral | 16.47 | 18.21 | 23.26 |
| 200708-9 | | diversity | isolate | | | |
| DAL | Dallas, TX | Geographic | Urethral | 20.61 | 24.12 | 26.61 |
| 200708-13 | | diversity | isolate | | | |
| DAL | Dallas, TX | Geographic | Urethral | 22.02 | 23.94 | 26.23 |
| 200708-24 | | diversity | isolate | | | |
| DEN | Denver, CO | Geographic | Urethral | 16.81 | 18.56 | NT |
| 200805-1 | | diversity | isolate | | | |
| DEN | Denver, CO | Geographic | Urethral | 17.64 | 19.95 | NT |
| 200805-2 | | diversity | isolate | | | |
| DEN | Denver, CO | Geographic | Urethral | 17.44 | 19.08 | NT |
| 200805-3 | | diversity | isolate | | | |
| DEN | Denver, CO | Geographic | Urethral | 17.54 | 19.39 | NT |
| 200805-4 | | diversity | isolate | | | |
| DEN | Denver, CO | Geographic | Urethral | 18.23 | 19.78 | NT |
| 200805-5 | | diversity | isolate | | | |
| R.DOM | Dominican | Geographic | Genital | 18.95 | 20.11 | NT |
| 061 | Republic | diversity | isolate | | | |
| R.DOM | Dominican | Geographic | Genital | 17.34 | 19.89 | NT |
| 1271 | Republic | diversity | isolate | | | |
| HON | Honolulu, | Geographic | Urethral | 18.10 | 19.57 | NT |
| 200806-1 | HI | diversity | isolate | | | |
| HON | Honolulu, | Geographic | Urethral | 17.24 | 19.15 | NT |
| 200806-2 | HI | diversity | isolate | | | |
| HON | Honolulu, | Geographic | Urethral | 17.03 | 18.26 | NT |
| 200806-3 | HI | diversity | isolate | | | |
| HON | Honolulu, | Geographic | Urethral | 17.55 | 19.12 | NT |
| 200806-4 | HI | diversity | isolate | | | |
| HON | Honolulu, | Geographic | Urethral | 17.55 | 18.75 | NT |
| 200806-5 | HI | diversity | isolate | | | |
| Kenya | Kenya | Geographic | Cervical | 17.83 | 20.19 | NT |
| 3559 | | diversity | isolate | | | |
| LAX | Los | Geographic | Urethral | 17.16 | 18.91 | NT |
| 200805-1 | Angeles, CA | diversity | isolate | | | |
| LAX | Los | Geographic | Urethral | 16.75 | 18.18 | NT |
| 200805-2 | Angeles, CA | diversity | isolate | | | |
| LAX | Los | Geographic | Urethral | 18.00 | 19.56 | NT |
| 200805-3 | Angeles, CA | diversity | isolate | | | |
| LAX | Los | Geographic | Urethral | 22.21 | 23.70 | NT |
| 200805-4 | Angeles, CA | diversity | isolate | | | |
| LAX | Los | Geographic | Urethral | 16.84 | 18.61 | NT |
| 200805-5 | Angeles, CA | diversity | isolate | | | |
| 33050 | Netherlands | Geographic | Isolate | 22.60 | 24.84 | NT |
| | | diversity | containing | | | |
| | | | 3.2 Mdal | | | |
| | | | plasmid | | | |
| P114 | Philippines | Geographic | Cervical | 17.40 | 18.89 | NT |
| | | diversity | isolate; | | | |
| | | | ciprofloxacin | | | |
| | | | resistant | | | |
| P115 | Philippines | Geographic | Cervical | 18.46 | 20.45 | NT |
| | | diversity | isolate; | | | |
| | | | ciprofloxacin | | | |
| | | | resistant | | | |
| PHIL 1- | Philippines | Geographic | Cervical | 17.68 | 20.44 | NT |
| 003 | | diversity | isolate | | | |
| PITT | Pittsburgh, | Geographic | Cervical | 18.13 | 20.54 | NT |
| 8396 | PA | diversity | isolate | | | |
| PITT | Pittsburgh, | Geographic | Cervical | 18.45 | 19.67 | NT |
| 9107 | PA | diversity | isolate | | | |
| POR | Portland, | Geographic | Urethral | 17.59 | 19.15 | NT |
| 200805-1 | OR | diversity | isolate | | | |
| POR | Portland, | Geographic | Urethral | 19.23 | 20.18 | NT |
| 200805-2 | OR | diversity | isolate | | | |
| POR | Portland, | Geographic | Urethral | 17.47 | 19.18 | NT |
| 200805-3 | OR | diversity | isolate | | | |
| POR | Portland, | Geographic | Urethral | 16.39 | 18.38 | NT |
| 200805-4 | OR | diversity | isolate | | | |
| POR | Portland, | Geographic | Urethral | 17.69 | 19.35 | NT |
| 200805-5 | OR | diversity | isolate | | | |
| SFO | San | Geographic | Urethral | 20.40 | 23.02 | 35.50 |
| 200708-7 | Francisco, | diversity | isolate | | | |
| | CA | | | | | |
| SFO | San | Geographic | Urethral | 21.03 | 23.26 | 33.45 |
| 200708-10 | Francisco, | diversity | isolate | | | |
| | CA | | | | | |
| SFO | San | Geographic | Urethral | 20.42 | 24.01 | 24.117 |
| 200708-20 | Francisco, | diversity | isolate | | | |
| | CA | | | | | |
| SK 07- | Seattle, WA | Geographic | Urethral | 20.25 | 23.56 | 26.52 |
| 325 | | diversity | isolate | | | |
| SK 07- | Seattle, WA | Geographic | Urethral | 19.89 | 23.49 | NT |
| 348 | | diversity | isolate | | | |
| SK 8-132 | Seattle, WA | Geographic | Urethral | 18.19 | 18.92 | NT |
| | | diversity | isolate | | | |
| SK 8-135 | Seattle, WA | Geographic | Urethral | 16.43 | 18.26 | NT |
| | | diversity | isolate | | | |
| SK 8-136 | Seattle, WA | Geographic | Urethral | 16.59 | 18.61 | NT |
| | | diversity | isolate | | | |
| SK 8-137 | Seattle, WA | Geographic | Urethral | 17.96 | 19.02 | NT |
| | | diversity | isolate | | | |
| SK 8-140 | Seattle, WA | Geographic | Urethral | 17.11 | 18.99 | NT |
| | | diversity | isolate | | | |
| G-172 | Senegal | Geographic | Cervical | 17.89 | 19.68 | NT |
| | | diversity | isolate | | | |
| P-301 | Senegal | Geographic | Cervical | 18.42 | 19.65 | NT |
| | | diversity | isolate | | | |
| SK-93- | Seattle, WA | Asymptomatic | Pharyngeal | 17.86 | 21.00 | 22.59 |
| 617 | | infection | isolate; AHU | | | |
| | | | requirements | | | |
| SK 97-10 | Seattle, WA | Asymptomatic | Urethral | 19.87 | 19.67 | 27.71 |
| | | infection | isolate; CU | | | |
| | | | requirements | | | |
| SK 97-12 | Seattle, WA | Asymptomatic | Urethral | 21.38 | 23.95 | 27.41 |
| | | infection | isolate; CU | | | |
| | | | requirements | | | |
| SK 97-3 | Seattle, WA | Asymptomatic | Urethral | 17.86 | 21.01 | 26.42 |
| | | infection | isolate; CU | | | |
| | | | requirements | | | |
| DAN | Denmark | opa mutations | Well- | 16.58 | 19.94 | 21.62 |
| 7823 | | | characterized | | | |
| | | | isolate with | | | |
| | | | opa | | | |
| | | | mutations | | | |
| DAN | Denmark | opa mutations | Well- | 17.56 | 18.87 | 21.08 |
| 7894 | | | characterized | | | |
| | | | isolate with | | | |
| | | | opa | | | |
| | | | mutations | | | |
| DAN | Denmark | opa mutations | Well- | 18.48 | 19.31 | 22.21 |
| 7895 | | | characterized | | | |
| | | | isolate with | | | |
| | | | opa | | | |
| | | | mutations | | | |
| DAN | Denmark | opa mutations | Well- | 16.27 | 19.11 | 22.61 |
| 7896 | | | characterized | | | |
| | | | isolate with | | | |
| | | | opa | | | |
| | | | mutations | | | |
| DAN | Denmark | opa mutations | Well- | 17.19 | 19.44 | 20.63 |
| 7901 | | | characterized | | | |
| | | | isolate with | | | |
| | | | opa | | | |
| | | | mutations | | | |
| ATCC | Georgia | Run to run control | Type strain | 21.20 | 23.80 | NT |
| 49226 | | | in many | | | |
| | | | settings | | | |

TABLE 5
SPECIFICITY TESTING OF NEAR NEIGHBOR ORGANISMS

| NRL ID | Species | opa Ct | porA Ct | Pil E Ct |
|---|---|---|---|---|
| 30018 | B. catarralis | undetected | 39.96 | NT |
| 30063 | B. catarralis | 34.84 | 34.91 | NT |
| 30069 | B. catarralis | undetected | undetected | NT |
| 30072 | B. catarralis | undetected | undetected | NT |
| 30524 | B. catarralis | 36.05 | 34.46 | NT |
| 30026 | N. animalis | undetected | 39.66 | NT |
| 30036 | N. animalis | undetected | 39.60 | NT |
| 30125 | N. animalis | undetected | undetected | NT |
| 30126 | N. animalis | undetected | undetected | NT |
| 30001 | N. canis | undetected | undetected | NT |
| 30003 | N. cinerea | undetected | undetected | NT |
| 30065 | N. cinerea | 33.93 | 37.36 | NT |
| 30066 | N. cinerea | undetected | undetected | NT |
| 32165 | N. cinerea | 40.00 | undetected | NT |
| 30005 | N. denitrificans | 37.75 | undetected | NT |
| 30006 | N. elongata | undetected | undetected | NT |
| 30046 | N. flava | undetected | 39.58 | NT |
| 30009 | N. flavescens | 35.26 | 35.66 | NT |
| 23970 | N. lactamica | 34.71 | 35.96 | NT |
| 30011 | N. lactamica | undetected | undetected | NT |
| 30022 | N. lactamica | 24.35 | undetected | NT |
| 30021 | N. meningitidis | undetected | undetected | NT |
| 9205 | N. meningitidis A | 39.01 | 37.18 | NT |
| 13090 | N. meningitidis B | undetected | 34.75 | NT |
| 9207 | N. meningitidis C | undetected | 38.71 | NT |
| 30013 | N. mucosa | 33.45 | 34.40 | NT |
| 30133 | N. mucosa | undetected | undetected | NT |
| | [Heidelbergenesis] | | | |
| 30047 | N. ovis | 36.05 | 38.47 | NT |
| 30015 | N. perflava | 33.94 | undetected | NT |
| 9913 | N. sicca | 34.35 | 39.18 | NT |
| 30016 | N. sicca | undetected | undetected | NT |
| 30045 | N. sicca | undetected | undetected | NT |
| 30052 | N. sicca | undetected | undetected | NT |
| 30056 | N. sicca | undetected | undetected | NT |
| 30017 | N. subflava | undetected | undetected | NT |
| 30044 | N. subflava | 35.11 | 35.68 | NT |

### Example 5

This example illustrates the analytical sensitivity of the assays.

Methods:

A control gonococcal strain ATCC 49226 was prepared in 10-fold serial dilutions from ˜10⁶ to 10¹ colony forming units/ml. Aliquots of each dilution were inoculated onto gonococcal base medium to confirm the quantity of organisms after 18 hours of growth. Extracted DNA from each concentration aliquot was tested in each assay/format combination. All tests were performed without knowledge of the concentration of the bacterial isolates.

In the por A assay, all 10-fold dilutions of ATCC 49226 were detected at Ct values ranging from 21.1 (10̂6 cfu/m) to 26.2 (10̂1 cfu/ml). Ct values and estimated CFU counts were correlated (R'0.829, Spearman's Rho). In the opa assay, all 10-fold dilutions of ATCC 49226 were detected at Ct values ranging from 18.93 (10̂6 cfu/ml) to 26.54 (10̂1 cfu/ml).

The analytic sensitivity of both the por A and opa assays is good: Detection was possible in specimens containing material that had yielded 1 colony-forming unit in culture.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for determining the presence of Neisseria gonorrhoeae in a test sample, said method comprising the steps of:
(a) contacting a test sample with a composition comprising at least one primer pair comprising a forward primer and a reverse primer capable of hybridizing to a target region of the N. gonorrhoeae opacity gene consisting of SEQ ID NO:2 to form a reaction mixture; and
(b) subjecting said reaction mixture to amplification conditions suitable to amplify at least a portion of said target region.

2. The method of claim 1, further comprising detecting the presence of the amplified portion by contacting the reaction mixture with a detection probe under hybridizing conditions, wherein the detection probe has a nucleotide sequence that hybridizes to at least a portion of the amplified target region and determining the presence of a hybrid.

3. The method of claim 1, wherein the composition comprises a primer having a target-binding region consisting of SEQ ID NO:3.

4. The method of claim 1, wherein the composition comprises a primer having a target-binding region consisting of SEQ ID NO:4.

5. The method of claim 2, wherein the detection probe comprises a target-binding region consisting of SEQ ID NO:5.

6. The method of claim 2, wherein determining the presence of said hybrid in said reaction mixture indicates the presence of N. gonorrhoeae in said test sample.

7. A method for determining the presence of N. gonorrhoeae in a test sample, said method comprising the steps of:
(a) contacting a test sample with a composition comprising at least one primer pair comprising a forward primer and a reverse primer capable of hybridizing to a target region of the N. gonorrhoeae porin A pseudo-gene consisting of SEQ ID NO:7 to form a reaction mixture; and
(b) subjecting said reaction mixture to amplification conditions suitable to amplify at least a portion of said target region.

8. The method of claim 7, further comprising detecting the presence of the amplified portion by contacting the reaction mixture with a detection probe under hybridizing conditions, wherein the detection probe has a nucleotide sequence that hybridizes to at least a portion of the amplified target region and determining the presence of a hybrid.

9. The method of claim 7, wherein the composition comprises a primer having a target-binding region consisting of SEQ ID NO:8.

10. The method of claim 7, wherein the composition comprises a primer having a target-binding region consisting of SEQ ID NO:9.

11. The method of claim 8, wherein the detection probe comprises a target-binding region consisting of SEQ ID NO:10.

12. The method of claim 8, wherein determining the presence of said hybrid in said reaction mixture indicates the presence of N. gonorrhoeae in said test sample.

13. A method for determining the presence of N. gonorrhoeae in a test sample, said method comprising the steps of:
(a) contacting a test sample with a composition comprising at least one primer pair comprising a forward primer and a reverse primer capable of hybridizing to a target region of the N. gonorrhoeae pilin E gene consisting of SEQ ID NO:12 to form a reaction mixture; and
(b) subjecting said reaction mixture to amplification conditions suitable to amplify at least a portion of said target region.

14. The method of claim 13, further comprising detecting the presence of the amplified portion by contacting the reaction mixture with a detection probe under hybridizing conditions, wherein the detection probe has a nucleotide sequence that hybridizes to at least a portion of the amplified target region and determining the presence of a hybrid.

15. The method of claim 13, wherein the composition comprises a primer having a target-binding region consisting of SEQ ID NO:13.

16. The method of claim 13, wherein the composition comprises a primer having a target-binding region consisting of SEQ ID NO:14.

17. The method of claim 14, wherein the detection probe comprises a target-binding region consisting of SEQ ID NO:15.

18. The method of claim 14, wherein determining the presence of said hybrid in said reaction mixture indicates the presence of N. gonorrhoeae in said test sample.

19. A method for determining the presence of N. gonorrhoeae in a test sample, said method comprising the steps of:
(a) contacting a test sample with a composition comprising at least one primer pair comprising a forward primer and a reverse primer capable of hybridizing to a target region of the N. gonorrhoeae opacity gene consisting of SEQ ID NO:2 and at least one primer pair comprising a forward primer and a reverse primer capable of hybridizing to a target region of the N. gonorrhoeae porin A pseudo-gene consisting of SEQ ID NO:7 to form a reaction mixture; and
(b) subjecting said reaction mixture to amplification conditions suitable to amplify at least a portion of said target region.

20. A method for determining the presence of N. gonorrhoeae in a test sample, said method comprising the steps of:
(a) contacting a test sample with a composition comprising at least one primer pair comprising a forward primer and a reverse primer capable of hybridizing to a target region of the N. gonorrhoeae opacity gene consisting of SEQ ID NO:2 and at least one primer pair comprising a forward primer and a reverse primer capable of hybridizing to a target region of the N. gonorrhoeae pilin E gene consisting of SEQ ID NO:12 to form a reaction mixture; and
(b) subjecting said reaction mixture to amplification conditions suitable to amplify at least a portion of said target region.

21. A method for determining the presence of N. gonorrhoeae in a test sample, said method comprising the steps of:
(a) contacting a test sample with a composition comprising at least one primer pair comprising a forward primer and a reverse primer capable of hybridizing to a target region of the N. gonorrhoeae porin A pseudo-gene consisting of SEQ ID NO:7 and at least one primer pair comprising a forward primer and a reverse primer capable of hybridizing to a target region of the N. gonorrhoeae pilin E gene consisting of SEQ ID NO:12 to form a reaction mixture; and
(b) subjecting said reaction mixture to amplification conditions suitable to amplify at least a portion of said target region.

22. A method for determining the presence of N. gonorrhoeae in a test sample, said method comprising the steps of:
(a) contacting a test sample with a composition comprising at least one primer pair comprising a forward primer and a reverse primer capable of hybridizing to a target region of the N. gonorrhoeae opacity gene consisting of SEQ ID NO:2, at least one primer pair comprising a forward primer and a reverse primer capable of hybridizing to a target region of the N. gonorrhoeae porin A pseudo-gene consisting of SEQ ID NO:7, and at least one primer pair comprising a forward primer and a reverse primer capable of hybridizing to a target region of the N. gonorrhoeae pilin E gene consisting of SEQ ID NO:12 to form a reaction mixture; and
(b) subjecting said reaction mixture to amplification conditions suitable to amplify at least a portion of said target region.

23. A set of oligonucleotides for use in amplifying a target region of nucleic acid derived from the opacity gene of N. gonorrhoeae, the set of oligonucleotides comprising a forward primer and a reverse primer, each primer having a target binding region up to 30 nucleotides in length, which contains at least 10 contiguous nucleotides that are perfectly complementary to an at least 10 contiguous nucleotide region present in a target sequence consisting of SEQ ID NO:2.

24. The set of oligonucleotides of claim 23, wherein the forward primer consists of SEQ ID NO:3.

25. The set of oligonucleotides of claim 23, wherein the reverse primer consists of SEQ ID NO:4.

26. The set of oligonucleotides of claim 23, further comprising a detection probe consisting of SEQ ID NO:5.

27. A set of oligonucleotides for use in amplifying a target region of nucleic acid derived from the porin A pseudo-gene of N. gonorrhoeae, the set of oligonucleotides comprising a forward primer and a reverse primer, each primer having a target binding region up to 30 nucleotides in length, which contains at least 10 contiguous nucleotides that are perfectly complementary to an at least 10 contiguous nucleotide region present in a target sequence consisting of SEQ ID NO:7.

28. The set of oligonucleotides of claim 27, wherein the forward primer consists of SEQ ID NO:8.

29. The set of oligonucleotides of claim 27, wherein the reverse primer consists of SEQ ID NO:9.

30. The set of oligonucleotides of claim 27, further comprising a detection probe consisting of SEQ ID NO:10.

31. A set of oligonucleotides for use in amplifying a target region of nucleic acid derived from the pilin E gene of N. gonorrhoeae, the set of oligonucleotides comprising a forward primer and a reverse primer, each primer having a target binding region up to 30 nucleotides in length, which contains at least 10 contiguous nucleotides that are perfectly complementary to an at least 1.0 contiguous nucleotide region present in a target sequence consisting of SEQ ID NO:12.

32. The set of oligonucleotides of claim 31, wherein the forward primer consists of SEQ ID NO:13.

33. The set of oligonucleotides of claim 31, wherein the reverse primer consists of SEQ ID NO:14.

34. The set of oligonucleotides of claim 31, further comprising a detection probe consisting of SEQ ID NO:15.

35. An oligonucleotide for use in amplifying a target region of nucleic acid derived from N. gonorrhoeae, said oligonucleotide having a target binding region of up to 30 bases in length, which stably hybridizes to a target sequence selected from the group consisting of SEQ ID NO:2, SEQ ID NO:7, and SEQ ID NO:12.

36. The oligonucleotide of claim 35, wherein said target binding region contains at least 10 contiguous nucleotides that are perfectly complementary to at least 10 contiguous nucleotides in said target sequence.

37. The oligonucleotide of claim 35, wherein said target region consists of SEQ ID NO:2.

38. The oligonucleotide of claim 37, wherein said target binding region consists of SEQ ID NO:3, SEQ ID NO:4, or SEQ ID NO:5.

39. The oligonucleotide of claim 35, wherein said target region consists of SEQ ID NO:7.

40. The oligonucleotide of claim 39, wherein said target binding region consists of SEQ ID NO:8, SEQ ID NO:9, or SEQ ID NO:10.

41. The oligonucleotide of claim 35, wherein said target region consists of SEQ ID NO:12.

42. The oligonucleotide of claim 41, wherein said target binding region consists of SEQ ID NO:13, SEQ ID NO:14, or SEQ ID NO:15.

43. A kit for detecting the presence of N. gonorrhoeae in a test sample, the kit comprising:
(a) at least one oligonucleotide comprising a target binding region sequence selected from the group consisting of SEQ ID NO:3, SEQ ID NO:4, SEQ ID NO:5, SEQ ID NO:8, SEQ ID NO:9, SEQ ID NO:10, SEQ ID NO:13, SEQ ID NO:14, and SEQ ID NO:15;
(b) amplification reagents; and
(c) written instructions describing amplification conditions suitable to detect the presence of N. gonorrhoeae in a test sample in the test sample.

44. The kit of claim 43, wherein one or more of the oligonucleotides incorporates one or more detectable labels.


SEQUENCE LISTING (15 sequences)

SEQ ID NO: 1 - DNA, 1434 bp, Neisseria gonorrhoeae
caggaacggcttggcacgttacagctacaccggttccaaacagtacggcaaccacagcggacaaatcggcgtaggctaccggttctgacggacagaaaacagacagccgcaaagatcacggtctttgcggctgtttcttatgaaaagaaaaccctattccaattgcctgcttctattgtttcaagacttcttccaaagattcggcatcaatcagacgtatagcggattaacaaaaatcaggacaaggggcgggccgcaggcagtacggatggtacggaaccgatccgcccgtgcttcagcaccttagggaaccgttccctttgagccggggcggggcaacgccgtaccggtttttgttcatccgccatattgtgttgaaacaccgcccggaacccgatataatccgcccttcaacatcagtgaaaatctttttttaaccggtcaaaccgaataaggagccgaaaatgaatccagcccgcaaaaaaccttctcttctcttctcttctcttctcttctcttctcttccgcagcgcaggcggcaagtgaaggcaatggccgcggcccgtatgtgcaggcggatttagcctacgccgccgaacgcattacccacgattatccggaaccaaccggtgcaaaaaaaggcacaacaataagcacggtaagcgattatttcagaaacatccgtacgcattccatccacccccgggtgtcggtcggctacgacttcggcggctggaggatagcggcagattatgcccgttacagaaagtggaacaacaataaatattccgtgagcataaaagagttgctaagaaacaagggcaatggcaacaggacagacctgaaggcggaaaatcaggaaaacggtacgttccacgccgtttcttctctcggcttgtccgccgtttacgacttcaaactcaacgataaattcaaaccctatatcggcgcgcgcgtcgcctacggacacgtcagacacagcatcgattcgaccaaaaaaacaacagaggttactaccatcctccatggtcctggcacaacccctacggtttatcctgggaaaaatacgcaaaacgcccatcgcgaaagcgacagcatccgccgcgtgggcctcggcgcagtggcaggcgtaggcatcgacatcacgcccaacctgaccttggacgccgggtaccgctaccactattggggacgcctggaaaacacccgcttcaaaacccacgaagcctcattgggcgtgcgctaccgcttctgattccccgataccgatgccgtctgaaccttcagacggcatttttaaatgcgcccgccgtttacaggcgcggggcgggcgcagtaaaatacccgaaccgtcattgccgatacaccgcaaccctaaaacccgccattcccgcgcaggggaatccggacctgtcgcgcggaaacttatcggat

SEQ ID NO: 2 - DNA, 229 bp, Neisseria gonorrhoeae
ataaggagccgaaaatgaatccagcccgcaaaaaaccttctcttctcttctcttctcttctcttctcttctcttccgcagcgcaggcggcaagtgaaggcaatggccgcggcccgtatgtgcaggcggatttagcctacgccgccgaacgcattacccacgattatccggaaccaaccggtgcaaaaaaaggcacaacaataagcacggtaagcgattatttcagaaac

SEQ ID NO: 3 - DNA, 25 bp, Artificial Sequence
  Synthetic
ataaggagccgaaaatgaatccagc

SEQ ID NO: 4 - DNA, 24 bp, Artificial Sequence
  Synthetic
gtttctgaaataatcgcttaccgt

SEQ ID NO: 5 - DNA, 15 bp, Artificial Sequence
  Synthetic
atgtgcaggcggatt

SEQ ID NO: 6 - DNA, 1332 bp, Neisseria gonorrhoeae
aaggtctgtatttaaatcatgttgcgggaaagcaacattttcaaaaaagttaatttattgttttatattgaaatattatttttcaaaataaaaattccaaaatttacccgaaatttgttccgaaaaatggtttttttttttcggggggtaattggagactgattgggtgtttgcccgatgtttttagcaaatttacaaaaggaagccgatatgcgaaaaaaacttaccgccctcgtattgtccgcactgccgtttgcggcagttgccgatgtcagcctgtacggcgaagtcaaagctggtgtggaaggcaggaacatccggctgcagttgaccgagccaccctcagaaggtcaaacgggcaatacagttactaaggccaaaagccgcatcaggacgaaagtcagtgatttcggctcgtttatcggctttaagggggtggggatttgggcggcgggctgaaggctgtttggcagctcgagcaagacgtatccgttgccggcggcggcgcgacccgttggggtaacagggaatcctttatcggcttggcaggcgaattcggcacggcgctcgccggtcgcgttgcgaatccgtttggcgatgccagcaaagccattgatccttgggacagcaataataatgtggcttcgcaattgggtattttcaaacgccacgacggtatgccggtttccgtgcgttacgattcccccggattttccggtttcagcggcagcattcaatttgttccgagtcaaaacagcaagtccgcctatacgcctgctactttcacgctggaaagtaatcagatgaaaccagttccggctgttgtcggcaagccggggtcggatgtgtattatgccggtctgaattacaaaaatggcggctttttcggaaattatgcccttaaatatgcgaaacacgccaatgaggggcatgatgctttctttttgttcttgctcggcagagcgagtgataccgatccattgaaaaaccatcaggtacaccgcctgacgggcggctatggggaaggcggcttgaatctcgccttggcggctcagttggatttgtctgaaaatgccgacaaaaccaaaaacagtacgaccgaaattgccgccactgcttcctaccgcttcggtaatacagtcccgcgcatcagctatgcccatggtttcgactttgtcgaacgcagtcagaaacgcgaacataccagctatgatcaaatcatcgccggtgtcgattacgatttttccaagcgcacttccgccatcatgtctgccgcttggctgaaacgaaataccggcatcggcaactacactcaaattaatgccgcctccg

SEQ ID NO: 7 - DNA, 77 bp, Neisseria gonorrhoeae
gggcatgatgctttctttttgttcttgctcggcagagcgagtgataccgatccattgaaaaaccatcaggtacaccg

SEQ ID NO: 8 - DNA, 21 bp, Artificial Sequence
  Synthetic
gggcatgatgctttctttttg

SEQ ID NO: 9 - DNA, 22 bp, Artificial Sequence
  Synthetic
cggtgtacctgatggtttttca

SEQ ID NO: 10 - DNA, 17 bp, Artificial Sequence
  Synthetic
cgagtgataccgatcca

SEQ ID NO: 11 - DNA, 507 bp, Neisseria gonorrhoeae
atgaatacccttcaaaaaggctttacccttatcgagctgatgattgtgatcgctatcgtcggcattttggcggcagtcgcccttcccgcctaccaagactacaccgcccgcgcgcaagtttccgaagccatccttttggccgaaggtcaaaaatcagccgtcaccgagtattacctgaataacggcatatggccggaaaagaacacttccgccggcgtggcatcctccgcctccgacatcaaaggcaaatatgttaaaagcgttgcagttgcaaacggcgtcgttaccgccacaatgctttcaagcggcgtaaacaatgaaatcaaaggcaaaaaactctccctgtgggccaagcgtcaagacggttcggtaaaatggttctgcggacagccggttacgcgcaccggcgacttcgacgacaccgttgccgacgccaaagacagcaaagaaatcgacaccaagcacctgccgtcaacctgccgcgataaggcatctgatgccaaatga

SEQ ID NO: 12 - DNA, 152 bp, Neisseria gonorrhoeae
gcaagtttccgaagccatccttttggccgaaggtcaaaaatcagccgtcaccgagtattacctgaataacggcatatggccggaaaagaacacttccgccggcgtggcatcctccgcctccgacatcaaaggcaaatatgttaaaagcgttg

SEQ ID NO: 13 - DNA, 21 bp, Artificial Sequence
  Synthetic
gsaagtttccgaagccatcct

SEQ ID NO: 14 - DNA, 28 bp, Artificial Sequence
  Synthetic
yaacbyyytbaacatatttgcctttgat

SEQ ID NO: 15 - DNA, 30 bp, Artificial Sequence
  Synthetic
acggcrhatggccsrvmvavaacvvykccg